(19) United States
(12) Patent Application Publication
(10) Pub. No.: US 2006/0177491 A1
(43) Pub. Date: Aug. 10, 2006

(54) Oil soft gel sheet for application to skin

(75) Inventor: Bong Jo Kim, Kuri-shi (KR)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Hanwoong Coatech Co., Ltd.

(21) Appl. No.: 11/349,856

(22) Filed: Feb. 7, 2006

(30) Foreign Application Priority Data
Feb. 7, 2005 (KR) 10-2005-11092

(51) Int. Cl. A61K 36/47; A61K 9/70; A61K 8/89

(52) U.S. Cl. 424/443; 424/70.12; 424/731

(57) ABSTRACT

The present invention relates to an oil soft gel sheet for application to the skin, which can be suitably used to allow an active ingredient to be absorbed into the skin or the human body through the skin. The oil soft gel sheet is in the form of a monolayer structure in view of cost versus effect and simple handling, has the release ability of a relatively large amount of oil components (including an active ingredient and its carrier), a suitable degree of skin adhesion, good stability with time, effect persistence after removal, and little skin stickiness after removal. The oil soft gel sheet is formed of a base comprising ethyl cellulose and one or two or more plasticizers, in which the weight ratio of ethyl cellulose to the plasticizers is 1-30:99-70. The plasticizer is preferably castor oil, and the base contains a component effective for the skin or human body, and may, if necessary, contain at least one selected from polyhydric alcohol, a surfactant and a silicon derivative.

### BACKGROUND

1. Field of the Invention

The present invention relates to an oil soft gel sheet for application to the skin that can be suitably used to allow an active ingredient to be absorbed into the skin or the human body through the skin.

2. Description of the Related Art

If an active ingredient (including a carrier thereof) is oil, it is used in the form of lotion, emulsion, ointments, or plasters. However, since lotion, emulsion, ointment, or the like are generally used by direct application to the skin with a finger or used in the form of, e.g., a gauze impregnated or coated with it, it cannot seem to be hygienic and requires efforts, and in some cases, it can be stuck to clothing, etc., so that the effect thereof cannot be sufficiently exhibited. To solve these problems, plasters have been proposed and, as the base thereof, an aqueous base, an oil base, and an emulsion base are used.

The aqueous base generally has a low content of an active ingredient (also including the carrier thereof) and is difficult to be formed into a thin layer. Also, the oil and emulsion bases contain rubber or synthetic resin as a main raw material and thus have an insufficient content of an active ingredient. Altogether, it is believed that the cohesion of the main raw material of the aqueous base is strong from the viewpoint of adhesion maintenance and stability with time and the aqueous base is hard gel, and its oil component is physically contained in the base and slowly moves in the base.

Thus the oil and emulsion bases are of practical use when the release amount of required oil components (including an active ingredient and its carrier) is very small or becomes the rate-determining step in absorption into the skin barrier, but they cannot cope with the case where the release amount of required oil components (including an active ingredient and its carrier) is relatively large or becomes the rate-determining step.

In the latter case, a drug delivery system having a more precise structure rather than a monolayer structure as in the present invention must be used, although it has increased cost and complexity in use. However, there are many cases where the application of the drug delivery system having a precise structure is not suitable in view of cost versus effect.

For example, the case of a local anesthetic drug that is used to relive itching and requires persistence, or the case of a keratin improvement drug for use on rough skin, relatively low cost should be first realized.

Also, users desire that, even after a plaster is removed, the effect thereof will be persistent to any degree. This is strongly desired also by medical technicians.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

It is an object of the present invention to provide an oil soft gel sheet for application to the skin that is in the form of a monolayer structure in view of cost versus effect and simple handling, has the release ability of a relatively large amount of oil components (including an active ingredient and its carrier), has a suitable degree of skin adhesion, has good stability with time, has effect persistence after removal, and has little skin stickiness after removal. Namely, an object of the present invention is to provide an oil soft gel sheet for application for the skin that has all advantages of ointments and plasters.

The present inventors have many studies to achieve the above object and, as a result, found that the object can be achieved by forming a sheet with a base comprising ethyl cellulose and one or two or more plasticizers, thereby completing the present invention.

Namely, the present inventors have performed studies on main materials including rubbers, plastics, celluloses, and hydrophilic polymers on the assumption that oil components (including an active ingredient and its carrier) need to be contained chemically other than physically in order to achieve the above object and, as a result, found that only a base comprising a plasticizer in addition to ethyl cellulose is suitable to achieve the above object. In this case, the area weight of the inventive sheet is preferably 30-3,000 g/cm² and, preferably, 100-2,000 g/cm². Also, in addition to the oil components (including an active ingredient and its carrier), the inventive sheet may, if necessary, contain an antioxidant, a pigment, a dye, a filler, and a fragrance. Moreover, the active ingredient may be hydrophilic in nature.

### DETAILED DESCRIPTION

In the sheet according to the present invention, the plasticizer can be selected from dibutylphthalate, castor oil, butyl stearate, and the like. Among them, castor oil is most preferable in view of the various properties of the sheet, including softness, skin adhesion, active ingredient release, stability with time, effect persistence and skin stickiness after removal, safety to the skin, production cost, and handling in production.

Of the above properties, the effect persistence after removal is particularly excellent when ethyl cellulose having a low degree of polymerization is used. This is believed to be because a portion of the base moves toward the skin to a suitable extent (i.e., an extent to which it does not cause skin stickiness) and remains around the skin wrinkles even after the removal of the sheet, so that it persistently releases oil components (including an active ingredient and its carrier).

The above composition consisting of ethyl cellulose and the plasticizer can be adjusted with regard to shape retention, release and skin adhesion by the polymerization degree of ethyl cellulose, the content of the placticizer, the mixing ratio between ethyl cellulose and the plasticizer, etc. The weight ratio of ethyl cellulose to the plasticizer is preferably 1-30:99-70.

However, there is a case where it is difficult to adjust effect persistence and skin stickiness by the physical properties of oil components (including an active ingredient and its carrier) and other additives. In this case, at least one selected from polyhydric alcohols, surfactants and silicon derivatives may be added for improvement. The most suitable and effective method is to add a combination of polyhydric alcohol and a silicon derivative.

Polyhydric alcohols that can be used in the present invention include glycerin, propylene glycol, and polyethylene glycol, and are used in an amount of 0.1-30% by weight based on the weight of the inventive composition for forming the sheet.

Surfactants which can be used in the present invention include anionic, cationic, non-ionic, and amphoteric surfactants and are used in an amount of 0.01-5% by weight based on the weight of the inventive composition for forming the sheet.

Suitable silicon derivatives which can be used in the present invention include a polyoxyethylene methylpolysiloxane copolymer, an alkylene glycol copolymer of silicon, and are used in an amount of 0.01-5% by weight based on the weight of the inventive composition for forming the sheet.

In the inventive sheet, a therapeutic drug or a beauty component is used as an active ingredient for application to the skin. Examples of the drug include all systemic drugs for in vivo absorption via the skin, including sterilizing/disinfecting agents, wound protective agents, agents for treating purulent diseases, analgesics, itching relieving agents, astringents, agents for treating parasitic skin diseases, skin softening agents, agents for hair, and hormonal agents.

Examples of the beauty component may include green tea and eucalyptus oil, which contain various nutrients, so that they serve as a beauty component by themselves and also has sterilizing performance.

In the case where the shape retention of the sheet itself is insufficient due to thin thickness, etc., the sheet may be stacked on a support member, such as a film or non-woven fabric made of, e.g., urethane or polyethylene. In the case where fixation to the skin needs to be increased, the sheet may be stacked on a top dressing including an adhesive layer on all or part of the surface thereof, the top dressing having an area larger than that of the sheet.

Hereinafter, the present invention will be described in detail by examples for a sheet for improving cracking and chapping of the heel skin, but the present invention is not limited in any way to these examples.

### EXAMPLES 1 TO 6 AND COMPARATIVE EXAMPLES 1 AND 2

### A) SAMPLES USED IN EXAMPLES

Ethyl cellulose and castor oil were first blended with each other by melting at 150° C., and then cooled to 100° C. Other components were added to the blended material to make a gel composition. The gel composition was coated on a 0.5 mm-thick urethane film at an area weight of 300 g/m². The resulting film was cut into a size of 5 cm×5 cm and the urethane film side was stuck to the center of the adhesive side of a non-woven fabric sheet having a size of 8 cm×8 cm that has been coated with an adhesive on one surface thereof.

The compositions of Examples 1 to 6, together with the compositions of Comparative Examples 1 and 2, are shown in Table 1 below.

TABLE 1

| | Example | Example | Example | Example | Example | Example | Comparative | Comparative |
|---|---|---|---|---|---|---|---|---|
| | 1 | 2 | 3 | 4 | 5 | 6 | Example 1 | Example 2 |
| Castor oil | 82.99 | 83.24 | 71.13 | 83.61 | 70.26 | 84.44 | Acrylic ester | — |
| Ethyl cellulose | 16.54 | 16.54 | 14.20 | 15.16 | 14.18 | 14.18 | PVA | — |
| PEG-12 dimethicone | — | — | — | 0.76 | 0.71 | — | Glycerin | — |
| PEG-60 hydrogenated | — | — | — | — | — | 0.71 | Urea | — |
| castor oil | | | | | | | | |
| Glycerin | — | — | 14.20 | — | 14.18 | — | Aloe vera extract | — |
| Eucalyptus oil | 0.10 | — | 0.10 | 0.10 | 0.10 | 0.10 | Crude rice bran extract | — |
| Green tea oil | 0.10 | — | 0.10 | 0.10 | 0.10 | 0.10 | Crude green tea extract | — |
| Mint oil | 0.05 | — | 0.05 | 0.05 | 0.05 | 0.05 | Squalene | — |
| V, E acetate | 0.10 | 0.10 | 0.10 | 0.10 | 0.10 | 0.10 | Citric acid | — |
| BHT | 0.10 | 0.10 | 0.10 | 0.10 | 0.10 | 0.10 | Lactic acid | — |
| 1.0 Red | 0.02 | 0.02 | 0.02 | 0.02 | 0.02 | 0.02 | Malic acid | — |
| Salicylic acid | — | — | — | — | 0.20 | 0.20 | Zinc oxide | — |
| Total | 100.00 | 100.00 | 100.00 | 100.00 | 100.00 | 100.00 | Paraben | — |

### B) SAMPLES USED IN COMPARATIVE EXAMPLES

In Comparative Example 1, a sheet for improving cracking and chapping of the heel skin, which is a commercially available product containing acrylic ester, squalene, and the like, was cut into a size of 5 cm×5 cm and stacked on the center of the adhesive surface of a non-woven fabric sheet having a size of 8 cm×8 cm that has been coated with an adhesive on one surface thereof. In Comparative Example 2, a non-woven fabric sheet having a size of 8 cm×8 cm that has been coated with an adhesive on one surface thereof, was used as it was.

### C) EVALUATION METHOD

The improvement degree of cracking and chapping of the heel skin was sensory evaluated by an evaluation group consisting of five persons.

Before retiring on Monday, different samples were applied on left and right heels, respectively, and in the morning on the next day, were removed. Upon the application, the samples were evaluated for skin adhesion, and at 1 hour after removal, were evaluated for skin stickiness felt by a finger and improvement effects. Then, upon bathing, the applied samples were lightly washed with soap one time every day for one week, and in the morning on the next day, whether improvement effects has been persistent was evaluated, in which an evaluation score of 3 or higher was estimated to be persistent for one day.

On Monday of the next week, two different samples were tested in the same manner as described above. However, since effects by test use can be cumulated to cause the skin condition to be changed (better), the heels were kept in severe conditions of dry humidity and low temperature after the end of persistence evaluation up to the next test day, such that the initial state of a fresh test could be maintained constant.

### D) EVALUATION ITEMS

Skin adhesion, skin stickiness, improvement effect, and persistence effect were evaluated.

### 1) Evaluation Criteria for Skin Adhesion

TABLE 2

| Evaluation score | Evaluation criteria |
|---|---|
| 1 | No stickiness felt with a finger |
| 2 | Weak stickiness felt with a finger |
| 3 | Slightly strong stickiness felt with a finger |
| 4 | Strong stickiness felt with a finger |
| 5 | Very strong stickiness felt with a finger |

In Table 2, evaluation scores of 3-5 are suitable for practical use and evaluation scores of 1-2 can be slippery upon application to the skin.

### 2) Evaluation Criteria for Stickiness of the Skin

TABLE 3

| Evaluation score | Evaluation criteria |
|---|---|
| 1 | No adhesion felt with a finger |
| 2 | Weak adhesion felt with a finger |
| 3 | A little strong adhesion felt with a finger |
| 4 | Strong adhesion felt with a finger |
| 5 | Very strong adhesion felt with a finger |

In Table 3, evaluation scores of 1-3 are suitable for practical use and evaluation scores of 1-2 are not of practical use.

### 3) Evaluation Criteria for Improvement Effect

TABLE 4

| Evaluation score | Evaluation criteria |
|---|---|
| 1 | Deteriorated |
| 2 | Changed |
| 3 | Improved |
| 4 | Significantly improved |

### 4) Evaluation Criteria for Effect Persistence

The number of persistent days was used as evaluation score. 2 days or longer were preferable.

### E) EVALUATION AND CONCLUSION

The evaluation results of the above tests are shown in Table 5 below.

TABLE 5

| | Example | Example | Example | Example | Example | Example | Comparative | Comparative |
|---|---|---|---|---|---|---|---|---|
| | 1 | 2 | 3 | 4 | 5 | \|6 | Example 1 | Example 2 |
| Skin adhesion | 4.2 | 3.0 | 3.2 | 2.8 | 3.0 | 3.4 | 4.2 | 1.0 |
| Skin stickiness | 5.0 | 2.8 | 2.6 | 2.2 | 1.0 | 2.8 | 1.0 | 1.0 |
| Improvement effect | 4.0 | 3.6 | 3.8 | 3.6 | 4.0 | 4.0 | 2.6 | 2.0 |
| Persistence | 3.0 | 2.8 | 2.8 | 2.2 | 3.0 | 2.8 | 0 | 0 |

From the above evaluation results, the following can be seen.

1. From the comparison between Examples 1-6 and Comparative Example 1, it can be seen that Examples have clearly significant differences in improvement effects and effect persistence.

2. From the results of Comparative Example 2 (top dressing itself), effects caused by the top dressing have little or no need to consider.

3. From the comparison of results between Examples 1 and 2, the effects of the additives can be seen.

4. From the results of Examples 3-6, it can be see that glycerin, PEG 60 hydrogenated castor oil, and PEF-12 dimethicone have the effect of inhibiting skin stickiness. Particularly, the combined use of glycerin and PEG-12 dimethicone is more effective.

As described above, unlike the prior products which are obtained by treating rubber or plastic resin with a solvent or heating the resin and contain no plasticizer, the sheet according to the present invention is prepared by treating ethyl cellulose with the plasticizer. Also, oil components (including an active ingredient and its carrier) in the inventive sheet are chemically contained in the base, and thus their content is large and their release property is excellent.

Also, since a part of gel in the inventive sheet migrates toward the skin to an extent to which it does not cause skin stickiness, the inventive sheet shows persistence even after removal. Namely, the present invention provides the oil soft gel sheet for application to the skin, which has all advantages of ointments and plasters.

While illustrative embodiments have been illustrated and described, it will be appreciated that various changes can be made therein without departing from the spirit and scope of the invention.

The embodiments of the invention in which an exclusive property or privilege is claimed are defined as follows:

1. An oil soft gel sheet for application to the skin, which is formed of a base comprising ethyl cellulose and one or two or more plasticizers.

2. The oil soft gel sheet of claim 1, wherein the plasticizer is castor oil.

3. The oil soft gel sheet of claim 1, wherein the base additionally contains at least one selected from polyhydric alcohol, a surfactant and a silicon derivative.

4. The oil soft gel sheet of claim 1, wherein the silicon derivative is an alkylene glycol copolymer of silicon.

5. The oil soft gel sheet of claim 1, which contains a component effective for the skin or human body.

6. The oil soft gel sheet of claim 2, which contains a component effective for the skin or human body.

7. The oil soft gel sheet of claim 3, which contains a component effective for the skin or human body.

8. The oil soft gel sheet of claim 1, which is stacked on a support member.

9. The oil soft gel sheet of claim 2, which is stacked on a support member.

10. The oil soft gel sheet of claim 3, which is stacked on a support member.
